(19) United States
(12) Patent Application Publication
(10) Pub. No.: US 2004/0031071 A1
(43) Pub. Date: Feb. 12, 2004

(54) SYSTEM OF HYSTEROSCOPIC INSEMINATION OF MARES

(75) Inventors: Lee H.A. Morris, Suffolk (GB); William R. Allen, Suffolk (GB); Allison C. Lindsey, Fort Collins, CO (US)

Correspondence Address: SANTANGELO LAW OFFICES, P.C., 125 SOUTH HOWES, THIRD FLOOR, FORT COLLINS, CO, 80521, US

(73) Assignee: XY, Inc., Fort Collins, CO (US)

(21) Appl. No.: 10/398,796

(22) Filed: Apr. 3, 2003

Related U.S. Application Data
(63) A 371 of international application No. PCT/US01/02304, filed on Jan. 24, 2001.
(63) Continuation of application No. 60/238,294, filed on Oct. 5, 2000.

(51) Int. Cl. C12N 15/00

(52) U.S. Cl. 800/21

(57) ABSTRACT

### Abstract of the Disclosure

The present invention provides a method of producing a mammal through artificial insemination and is directed, in particular embodiments, to low spermatozoa numbers for insemination and the production of a mammal through the use of hysteroscopic insemination techniques. The present invention is particularly directed to embodiments potentially regarding fresh or preserved sperm, treated or processed sperm, sperm inserted under a surface in the vicinity of the uterotubal junction, hysteroscopic compatible media for the establishment of the insemination sample, hysteroscopic compatible volume for insemination, epididymal use of the hysteroscopic technique, bubble or froth insemination utilized in hysteroscopic insemination, and for sorted and frozen sperm utilized in hysteroscopic insemination. The disclosed embodiments may be directed at a mammal species, particularly equids, bovids, and swine, as well as animals produced in accordance with any of the disclosed embodiments of the present invention.

### Detailed Description of the Invention

### Cross Reference to Related Applications

This application is the National Stage of International Application No. PCT/US01/02304, filed January 24, 2001, which claims the benefit of U.S. Provisional Application No. 60/238,294, filed October 5, 2000, each priority application hereby incorporated by reference.

### Background of Invention

This invention relates generally to the field of insemination of mammals. Specifically, it relates to systems to achieve insemination which may be particularly applicable once sperm have been treated or processed in some manner, such as sorting the sperm through flow cytometry. More particularly, the invention may relate to systems to achieve insemination, with a low number of spermatozoa as compared to conventional artificial insemination, through hysteroscopic insemination techniques. The invention may be particularly applicable to systems for inseminating equids, bovids and swine. Furthermore, the invention may be particularly applicable to sex selection of offspring in mammals.

Artificial insemination (AI), in various forms, has been used to achieve successful rates of pregnancy and resulting offspring in mammals. Traditional forms of AI include various in vivo technologies such as conventional intrauterine artificial insemination techniques, or more particularly, trans cervical artificial insemination. Natural insemination doses may typically be large. For example, in some species, natural insemination may involve sperm numbers on the order of 2-20 x 10⁹ sperm. In contrast, conventional AI techniques in the same species may require sperm numbers on the order of 200-500 x 10⁶ sperm. This can still be viewed as a large number of sperm, especially if the sperm are processed in some manner. Given the traditional requirements for high numbers of sperm to achieve acceptable numbers of pregnancies, other insemination techniques have been sought to address developing breeding technologies that may result in a lower sperm count for subsequent insemination. Low dose insemination has been particularly discussed in the PCT publication WO 99/33956, entitled "Sex-Specific Insemination of Mammals With Low Number of Sperm Cells." Hysteroscopic insemination has been proposed and to some extent discussed in Morris et al., 2000, hereby incorporated by reference.

It should be preliminarily noted that all references mentioned in this application, and including additional reference not mentioned, are listed at the end of this written discussion, all of which should be considered as incorporated by reference.

High-speed sperm sorting using flow cytometry has been used successfully as a breeding technology to produce offspring in mammals, such as horses, cattle, sheep, rabbits, pigs and humans. It can potentially be used for several other species as well. Technologies have been developed to enhance or modify the pregnancy and resulting offspring of mammals, particularly with regard to the processing of sperm and insemination techniques.

One potential limiting factor in optimizing the success of artificial insemination, as recognized in Pickett et al., 1989, may be inseminating with low numbers of spermatozoa. Pickett et al. recognized that a potential minimal recommended dose for conventional artificial insemination in the mare may be as high as 500 x 10⁶ progressively motile sperm. With low sort rates of around 700 spermatozoa per second in some sperm sorting technologies, it may take several days to obtain the recommended dose of spermatozoa for artificial insemination. This may not only be impractical, but the viability of the spermatozoa may also be significantly reduced. Low-dose insemination techniques, therefore, may be a desirability to those skilled in the art to potentially reduce the number of spermatozoa needed for acceptable fertility rates.

Additionally, what might be considered low doses for insemination may not typically result in acceptable fertility rates. Fertility rates may be considered acceptable or statistically comparable, for example, if they are achievable over a high sample size, range or percentage of the fertility rates resulting from conventional AI. Low-dose insemination techniques, therefore, may be a desirability to those skilled in the art to potentially reduce the number of spermatozoa needed to maximize fertility.

Whether in consideration of low-dose insemination or to improve AI fertility rates generally, an additional concern regarding artificial insemination is the efficiency of the procedure as a whole with regard to the resulting numbers of pregnancies. A number of procedural steps may have been used in conventional AI procedures, such as the synchronization of estrus in breeding mares; the preparation of the insemination dose, more particularly the use of extenders for the dilution (Kenny et al., 1975) and resuspension of spermatozoa (using TALP or HEPES-buffered Tyrode"s Medium, for example), centrifuging the sample through a density or viscosity gradient (using PERCOLL or the like); assessing viability or motility; and the particulars of the insemination procedure. Insemination procedures have historically included ultrasound and rectally-guided techniques. Hysteroscopic insemination of mares has also been conducted, as described in the Vasquez et al., 1998 and Manning et al., 1998 references. Only limited success, however, has been demonstrated in utilizing the above described procedures in conventional AI and in the Vasquez et al. and the Manning et al. procedures. In particular, neither reference may have produced statistically comparable conception rates to conventional AI. In particular, the Vasquez et al. and the Manning et al. references may have demonstrated conception rates of 33% for 3.8 x 10⁶ spermatozoa and 22% for 1.0 x 10⁶ spermatozoa, respectively, which may be considered non-comparable conception rates relative to conventional AI for the species involved. Despite the previous and substantial attempts at producing an efficient procedure for AI, heretofore, a long felt but unsatisfied need for an efficient procedure for the hysteroscopic insemination of mammals has existed in breeding technology. Furthermore, and given the traditional requirements for high numbers of spermatozoa to achieve acceptable numbers of viable pregnancies, as described above, a need for efficient, low-dose hysteroscopic insemination has heretofore existed in current breeding technology.

Furthermore, deep intra uterine insemination of swine has been conducted, as described in the Vasquez et al., 2000 reference. However, insemination was conducted with what might be considered a high number of spermatozoa, on the order of 20 x 10⁷ spermatozoa. However, Vasquez et al., 2000, may have identified the long felt but unsatisfied need for an insemination procedure to address breeding technologies utilizing lower spermatozoa numbers for insemination, such as may result from current sperm sorting technologies. Therefore, as may have been demonstrated from the Vasquez et al., 1998, Vasquez et al., 2000, and Manning et al., 1998, references, those skilled in the art may have failed to address the identified need for providing an insemination technique potentially resulting in high fertility rates in mammals and the resulting high production in mammals, high fertility rates for low insemination dosages, and insemination techniques to address issues of efficaciousness, particularly with regard to the hysteroscopic insemination technique.

One important procedural step with regard to insemination procedure, generally, is the establishment of a insemination dose containing desirable numbers of viable and motile spermatozoa to potentially provide higher fertility rates. Procedures for the selection of motile spermatozoa may have been conducted with regard to conventional AI, for example in the reference Grøndahl et al., 1996 and in hysteroscopic insemination generally, by establishing a density or viscosity gradient utilizing, for example, PERCOLL (Sigma Chemical Co., St. Louis, MO) alone or in combination with other substances. However, heretofore the substantial efforts to fractionate viable or motile sperm have not particularly addressed the identified needs for an insemination technique potentially providing high fertility rates, high fertility rates for low insemination dosages, and insemination techniques to address issues of efficaciousness, particularly with regard to the hysteroscopic insemination technique. A second potential issue with regard to insemination procedure, generally, is the establishment of a insemination dose containing desirable numbers of viable and motile spermatozoa to potentially provide higher fertility rates without a particular motility test, as described above. The introduction of a density or viscosity gradient may introduce a stress to the spermatozoa that may actually reduce the actual number of viable and motile spermatozoa available from a particular sample. Heretofore, the substantial efforts to fractionate viable or motile sperm have not particularly addressed the identified needs for an insemination technique providing for high fertility rates, high fertility rates for low insemination dosages, and insemination techniques to address issues of efficaciousness, particularly with regard to the hysteroscopic insemination technique. In fact, due to the potential for reduction in the total number of viable or motile sperm from a particular inseminate sample, previous attempts incorporating a density or viscosity gradient may have actually taught away from the present procedure of providing for an insemination technique which may require less efforts to obtain a viable, low-dose insemination sample. A third potential issue with regard to insemination procedure, generally, is the establishment of a compatible volume for the particular insemination technique. One recognized need, as described above, is the desire to potentially provide higher fertility rates. A second recognized need, also described above, is the ability to use low numbers of spermatozoa to potentially achieve high fertility rates. The insemination dose volume may be determined by the particular insemination technique. However, the dose volume may contain a desirable number of spermatozoa to potentially provide for a higher rate of fertility. Substantial attempts have been made to establish an appropriate insemination technique that would allow for the appropriate number of spermatozoa, given the potential volume requirements of the insemination technique, to potentially provide acceptable fertility rates. However, determining a compatible insemination dose volume for a particular insemination technique, to further potentially provide higher rates of fertility, has potentially not been established for hysteroscopic insemination, as the reported fertility rates in Vasquez et al. and Manning et al. may demonstrate.

Additionally, and as previously mentioned, the sperm sample may be processed prior to the insemination procedure. Conventional AI, for example, may provide for the use of extenders for the dilution (Kenny et al., 1975) and resuspension of spermatozoa. However, the particular media used may not be compatible with the insemination procedure itself. Incompatibility of the sample media may result in lower deposition numbers of spermatozoa or dose volume or a lower fertility rate. Furthermore, the mode or form of the deposited insemination dose or the particular method of deposit during AI may further affect the number of deposited spermatozoa available for conception. However, heretofore the substantial efforts directed toward insemination media may have not particularly addressed the need for providing an insemination technique potentially resulting in high fertility rates, high fertility rates for low insemination dosages, and insemination techniques to address issues of efficaciousness, particularly with regard to the hysteroscopic insemination technique.

An additional factor to consider is the timing of insemination. Insemination timing may be an important factor, for example, to sperm viability and longevity and the timing of the estrous cycle of the mammal. Particularly at issue might be the distant location of sperm sample acquisition (i.e. the location of the male mammal) and ultimate location of the AI. Previous efforts may have been made in conventional AI to preserve the sperm sample prior to insemination and to coordinate the insemination with the estrous cycle. However, heretofore the substantial efforts directed to insemination timing may have not particularly addressed the identified needs for, and may have even failed to understand the problems of, providing an insemination technique potentially resulting in high fertility rates, high fertility rates for low insemination dosages, and insemination techniques to address issues of efficaciousness, particularly with regard to the hysteroscopic insemination technique.

The source of the sperm sample may also be of importance to the resulting insemination. Epididymal acquisition of the sperm sample (obtaining sperm sample from the epididymis of the testis; ductules emerging posteriorly from the testis that holds sperm during maturation and that forms a tangled mass before uniting into a single coiled duct which is continuous with the vas deferens) may provide some inherent advantages as to timing of the insemination and viability of the sperm. However, heretofore the substantial efforts directed to sperm source, viability and insemination timing may have not fully addressed the identified needs for, and may have even failed to understand the problems of, providing an insemination technique potentially resulting in high fertility rates, high fertility rates for low insemination dosages, and insemination techniques to address issues of efficaciousness, particularly with regard to the hysteroscopic insemination technique.

### Summary of Invention

The present inventors have recognized the potential problems associated with conventional methods of artificial insemination. Accordingly, embodiments of the present invention may provide for the production of a mammal through the use of artificial insemination that may address inadequacies of previous insemination techniques and systems. The invention may comprise, according to particular embodiments, a method of producing a mammal whereby potentially high fertility rates may be accomplished, fertility rates which may be statistically compatible with conventional AI results, and potentially high fertility rates with the use of low spermatozoa doses. More particularly, embodiments of the present invention may provide for the production of a mammal through the use of hysteroscopic insemination techniques. Additionally, the present invention may comprise embodiments particularly directed at mammals such as equids, bovids, and swine, among other mammals. Embodiments of the present invention, therefore, may even be considered development away from previous efforts of artificial insemination.

One object of the present invention, therefore, is to provide for the production of a mammal utilizing an efficacious procedure. Therefore, a goal of the present invention is to provide a technique of artificial insemination for mammal production such that lower numbers of spermatozoa may be used in the insemination dose relative to conventional AI and other insemination techniques, while, in particular embodiments of the invention, at least statistically comparable success rates in fertility are maintained.

Another object of the present invention is to provide for the production of a mammal utilizing an artificial insemination procedure that may potentially achieve high fertility rates consistent with lower spermatozoa production from breeding technologies such as sperm sorting. A goal of the present invention, therefore, is to provide a technique of artificial insemination for mammal production that achieves statistically comparable success rates in fertility, compared to conventional AI and other insemination techniques, with lower-doses of spermatozoa.

Furthermore, an object of the invention is to provide for the production of a mammal utilizing an artificial insemination procedure that may enhance steps involved in the artificial insemination. To this end, one goal of the present invention is to provide a technique of artificial insemination for mammal production such that steps of estrous cycle timing, spermatozoa source, viability, longevity and processing, insemination dose media and volume, and insemination timing may be optimized, particularly for low-dose insemination and potentially high fertility.

An additional object of the present invention, according to preferred embodiments, is to provide for the production of a mammal through hysteroscopic insemination. A goal of the present invention, therefore, is to provide a technique of artificial insemination for mammal production such that the insertion of the insemination dose, guiding of the insemination dose to the deposition site, deposition of the insemination dose at the appropriate location, in an appropriate mode or form, may be accomplished to achieve other objects and goals as previously stated. In accordance with particular embodiments of the invention, blister insemination and bubble or froth insemination may be introduced as preferred embodiments to optimize fertility rates. A particular goal of the present invention is to provide a technique of artificial insemination for mammal production utilizing a catheter comprising a videoendoscope for guiding and depositing the insemination dose.

Additionally, an object of the present invention, in accordance with particular embodiments, is to provide for the production of various mammal species utilizing an artificial insemination procedure. A goal of the present invention, therefore, is to provide a technique of artificial insemination for mammals such as equids, bovids and swine, among other species. A further goal is to provide a technique of artificial insemination for various mammal species that additionally provides for low numbers of spermatozoa in the insemination dose and for potentially high fertility rates, particularly rates that may be statistically comparable to conventional AI.

Other objects of the invention are disclosed throughout other areas of the specification and claims. In addition, the goals and objectives may apply either in dependent or independent fashion to a variety of other goals and objectives in a variety of embodiments.

### Brief Description of Drawings

Figure 1 is a partially exploded and perspective diagrammatic view of the optical element, cannula, catheter and syringe in accordance with one embodiment of the present invention.

Figure 2 is a diagrammatic depiction of the reproductive organs of a female of a mammalian species and, in particular, a depiction of artificial insemination in accordance with one embodiment of the present invention.

Figure 3 is a magnified diagrammatic view of an enshrouded insemination insertion embodiment of the present invention.

### Detailed Description

The basic concepts of the invention may be embodied in many different ways. The inventive concept may involve the materials, elements, apparatus, device and methods for the production of a mammal through artificial insemination. Furthermore, while one preferred embodiment of the invention may be particularly directed to the production of equids through artificial insemination, the broad concept of the invention should be construed as a disclosure of the production of mammals in general, and as indicated, to other mammal species such as bovids and swine.

As should be understood, the present invention includes a variety of aspects that may be used in various combinations depending upon the application"s needs. The invention is intended to encompass a variety of embodiments of mammal production and combinations thereof. It involves both methods and devices to accomplish the various aspects explained. In addition, while some methods and devices are disclosed, it should be understood that these may be varied. Importantly, as to all of the foregoing, all aspects should be understood to be encompassed by this patent both independently and in combination as set forth in the claims now or later issued.

Accordingly, one embodiment of the present invention may provide for the collection of sperm cells from a male of the species of interest. In accordance with one embodiment, sperm cells are collected from one or more stallions of the equine species. According to this embodiment, semen may be collected, and in preferred embodiments, semen may be collected with a commercially available artificial vagina, perhaps from at least one stallion of known acceptable fertility. An artificial vagina such as one made available by Animal Reproduction Systems may be used with an in-line gel filter, and in preferred embodiments, used on alternate days throughout collection. After collection, the semen may be evaluated for gel-free volume, motility, and sperm concentration. In accordance with another preferred embodiment, sperm cells may be collected from other male species of mammal, particularly that of bovids, equids or swine. An alternative embodiment of the present invention may provide for the collecting of epididymal sperm cells obtained from the epididymis of the testis of the male species of the mammal. The alternative embodiment providing for the use of epididymal sperm may be incorporated with all other disclosed embodiments herein, either in single or in combination. Furthermore, the present invention provides particular embodiments a hysteroscopic insemination sample comprising a reservoir element, a catheter system to which the reservoir element is responsive, and a plurality of epididymal sperm cells contained within the reservoir element.

After sperm collection, an artificial insemination sample may be established for the insemination of the female species. In accordance with one embodiment of the present invention, the sample may be prepared as having a low number of sperm compared to a natural insemination dosage for the mammal. The sample may have a low number of sperm for particular breeding technologies, and in accordance with preferred embodiments, the sample may have a low number of sperm as the result of sorting the sperm for particular sexed sperm. In accordance with a preferred embodiment of the invention, the spermatozoa may be stained with Hoechst 33342 and sorted into X and Y chromosome-bearing populations based on DNA content using a commercially available SX MoFlo®sperm sorter.

Additionally, an artificial insemination sample may be established at volumes, in accordance with preferred embodiments, at volumesbetween about 30 and 150 ul, less than about 500 ul, about 230 ul, and about 100 ul. One embodiment of the present invention is directed to establishing a hysteroscopic insemination compatible volume, preferably an insemination sample at a volume selected from a group consisting of: between about 30 and 150 ul, less than about 500 ul, about 230 ul, and about 100 ul. Furthermore, the present invention is directed to a hysteroscopic insemination sample comprising a reservoir element a catheter system to which the reservoir element is responsive, and a hysteroscopic compatible volume of sperm contained within said reservoir element. The hysteroscopic compatible volume of sperm contained within said reservoir element may comprise a volume selected from a group consisting of: between about 30 and 150 ul, less than about 500 ul, about 230 ul, and about 100 ul.

Next, in accordance with preferred embodiments of the invention, the artificial insemination sample may be placed within a catheter or catheter system. In accordance with alternative embodiments, the sample may be placed within a reservoir element or other sample holding element responsive to the catheter or catheter system. A catheter or catheter system should be understood to define any device, system or method of insertion into canals, vessels, passageways, or body cavities to permit injection or withdrawal of fluids, and in accordance with preferred embodiments such injection or withdraw may provide the response of the reservoir element or sample holding element, or to keep a passage open.

Preferably, the catheter or catheter system may be used in conjunction with a guide element, and in preferred embodiments an optical element or device, and in preferred embodiments an illumination element, to provide guidance in the artificial insemination procedure, as more particularly described below. Preferred embodiments may provide the strobing of the illumination element. However, manual guidance may also be implemented. In accordance with preferred embodiments of the invention, and as depicted in Figure 1, the insemination sample may be aspirated into an equine GIFT catheter (2) (Cook Veterinary Products, Brisbane, Australia) using preferably a 6-mL disposable syringe (4) attached to the injection port (6) on the distal end (8) of the catheter. The loaded catheter may be withdrawn into a tube, or preferably an outer polypropylene cannula (10), which may be responsive to an optical element (12), and in accordance with preferred embodiments, passed down a working channel of a Pentax EPM 3000 videoendoscope (Pentax UK Ltd, Slough, Bucks UK).

Accordingly, an instance, time or duration may be determined in which the artificial insemination may be appropriate. In accordance with preferred embodiments of the present invention, a determination may be made as to an estrous time for a female mammal. In accordance with an embodiment of the invention, estrus may be induced to determine the estrous time and, for multiple mares, even synchronized. Estrus may be defined as a state in which the female mammal is capable of conceiving and estrous cycle may be defined as the correlated phenomena of the endocrine and generative systems of a female mammal, potentially from the beginning of one period of estrus to not later than the beginning of the next. In accordance with a preferred embodiment, estrus may be induced, and for multiple female mammals synchronized, by administering a substance such as a progestagen, preferably for mares altrenogest, and preferably 10ml orally, potentially for 10 consecutive days, followed by 250 µg cloprostenol i.m., potentially on day 11. Furthermore, a female mammal may be induced into ovulation at the time of insemination. Ovulation may be induced, and in preferred embodiments, by the administration of 3000 iu human Chorionic Gonadotropin (hCG, Chorulon, Intervet, Inc., Millsboro, Holland), preferably administered intravenously at the time of insemination or up to approximately 8 hours previously. Ovulation may even be induced, in preferred embodiments, by the administration of from about 2000 to about 5000 iu human Chorionic Gonadotropin.

In preferred embodiments, estrous cycles may be synchronized by administering a synthetic progestagen altrenogest (0.044mg/kg p.o., Regumate; Hoechst Roussel Vet, Warren, New Jersey, USA) daily for 10 consecutive days. Luteolysis may be induced, in preferred embodiments with the prostaglandin analogue, cloprostenol (250µg Estrumate, i.m.; Bayer Corporation, Agriculture Division, Shawnee Mission, Kansas, USA) administered on the eleventh day.

A time when the female mammal is appropriately fertile may be determined. The ovaries may be examined, and in preferred embodiments examined ultrasonographically, and preferably every second day until a follicle, and in accordance with preferred embodiments a dominant follicle, preferably of > 30mm diameter, is detected. The female may be examined until a follicle, preferably pre-ovulatory, of preferably > 35mm is detected. In one embodiment, the female may be inseminated during the same day as estrous inducement or synchronization, the same day as ovulation inducement or synchronization, or the same day as estrus and insemination inducement or sychronization.

Next, in accordance with particular embodiments of the invention, the guide element, and in preferred embodiments the optical element (12), may be vaginally inserted into the female. Furthermore, the catheter (2), and in preferred embodiments the reservoir element, may be inserted into the female. The sequence of insertion of the optical element (12) and the catheter (12) may be sequential or coincidental in time.

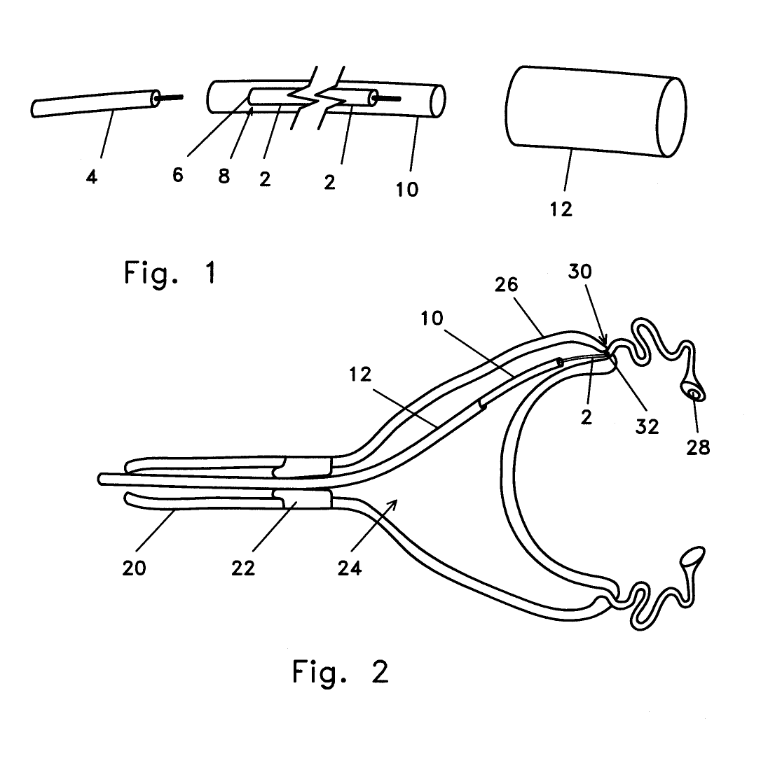

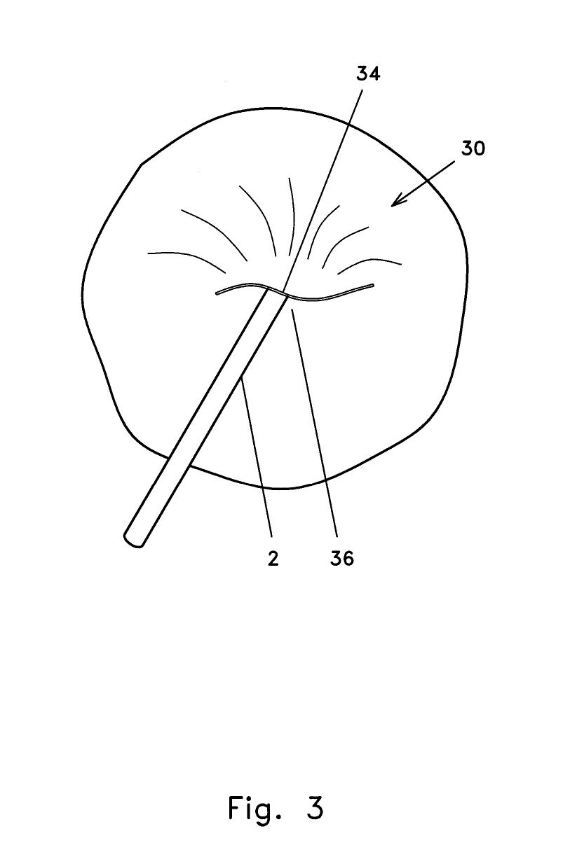

The guide element or optical element (12) and the catheter (2) may then be guided through the vagina (20) of the female, as depicted in Figure 2. In accordance with one embodiment, the optical element (12) and catheter (2) may be manually guided. In fact, the catheter (2) may be manually guided without the optical element (12). However, accuracy in finding the UTJ and the potential result in increased fertility rates, particularly for low numbers of sperm and overall potential efficacy in the procedure, may require a more accurate guidance procedure. In preferred embodiments, the optical element or endoscope, preferably a flexible endoscope, in preferred embodiments having dimensions of 1.6 m long with an outer diameter of 12 mm, may be guided through the cervix (22) and propelled forward through the uterine lumen (24), or in additional embodiments, through a uterine horn of the female of the species. An added benefit of the use of the videoendoscope can be lack of a need to rectally guide the insemination process, as may have been required in past efforts by those of skill in the art.

The uterotubal junction (UTJ) (30) of the female mammal may then be located, preferably optically with the optical element (12). The catheter (2) may then be positioned in the vicinity of or proximate to the uterotubal junction. In accordance with preferred embodiments, the endoscope may be directed under visual control along the uterine horn (26) ipsilateral to the ovary containing the pre-ovulatory follicle (28). Preferably the tip of the endoscope may be directed proximate to, and in preferred embodiments within about 3-5cm of said uterotubal junction, and in preferred embodiments within about 3-5 cm of the papilla (32) of the uterotubal junction.

At least a portion of the artificial insemination sample may then be extruded from the catheter (2). In accordance with preferred embodiments, the outer cannula (10), followed by the inner GIFT catheter (2) containing the sperm suspension, may be extruded from the working channel of the endoscope until the tip of the GIFT catheter touches the uterotubal junction, and in preferred embodiments, touches the papilla. In accordance with alternative embodiments of the present invention, a portion or at least a portion of the artificial insemination sample may be aspirated during extrusion from the catheter, thereby potentially creating an aspirated sample. Such aspirated samples should be understood to include bubbled samples and frothed samples. An aspirated sample may not only provide better adherence to a surface in the vicinity of the UTJ, but may further allow for improved fertility rates. Furthermore, an embodiment of the present invention provides a hysteroscopic insemination element comprising a uterus of a female species of a mammal, a plurality of sperm cells contained within said uterus, and an aspirated volume of media surrounding or interspersed with the sperm cells and to which the sperm cells are responsive. Such alternative embodiments should be construed as disclosed with regard to all embodiments of the present invention, either in single or in combination, and should be construed to be disclosed as such.

A low number of sperm may be deposited in the vicinity of the uterotubal junction. In accordance with preferred embodiments, a plunger of the syringe (4) may be depressed to deposit the sample, and in particular embodiments, a small volume (preferably perhaps ~100µl) of the sample, preferably onto the surface of the papilla. The guide element, and in preferred embodiments the optical element (12), and the catheter (2) may be withdrawn from the uterus of the female mammal. In accordance with preferred embodiments, filtered air may be introduced within the uterus of the female to facilitate passage of the instruments through the uterine lumen. Furthermore, in accordance with preferred embodiments, the filtered air may be evacuated from the uterus, preferably simultaneous to the withdrawal of the optical element (12) and catheter (2).

The placing of a low number of sperm may be accomplished, in one preferred embodiment of the invention, by placing with the catheter (2) a number of sperm, preferably numbers selected from: less than about ten million sperm, less than about five million sperm, less than about two million sperm, less than about one million sperm, less than about five hundred thousand sperm, and less than about one hundred thousand sperm.

Subsequently, the fertilization of an egg of the female may occur and the production of an offspring mammal from the fertilized egg. In accordance with preferred embodiments of the invention, success levels of fertilization may be statistically comparable to a conventional uterine body artificial insemination process. Statistically comparable success levels may be defined as fertilization rates of at least about 75% success rates, at least about 65% success rates, at least about 60% success rates, at least about 50% success rates, at least about 45% success rates, and at least about 90% of a success rate practically experienced with conventional AI for any particular species. Furthermore, success levels for the present invention may be statistically comparable to a conventional uterine body artificial insemination process over a sample of cumulative fertilizations which is greater than about 100, which is greater than about 500, and which is greater than about 1000. Success rates may further statistically provide for at least a confidence level of at least about 95 percent confidence (potentially expressed as P> or equal to about 0.05), therefore potentially being statistically comparable with conventional AI. Additionally, success rates for the present invention may potentially have the same P value over a range of differing sperm numbers, potentially such as 1, 5 or 10 x 10⁶ sperm for equine. Furthermore, a success level or rate may be expressed in terms of sample size, whereby the present invention may provide, in preferred embodiments, any of the aforementioned success rates over a power calculation (β) of at least about 80 percent. Additionally, the success rates, in preferred embodiments, given particular insemination doses for sex-sorted stallion spermatozoa, may even apply to a low number of spermatozoa and may routinely produce fertility rates of at least about 90% of those rates resulting from conventional artificial insemination for a species. Additionally, according to particular embodiments of the present invention, the previous success rates may even be achieved for species of mammal such as bovids, equids, and swine.

In regard to sperm viability, longevity and mobility, for example, the present invention may provide for establishing an artificial insemination sample utilizing fresh sperm. The term "fresh sperm" may be broadly defined as sperm that has not been treated, processed or preserved in any manner such that the sperm viability, longevity and/or mobility might be compromised. Such treatment, processing or preserving may include, for example, the sorting of sperm, the freezing and subsequent thawing of sperm, the dilution and resuspension of sperm, and motility and viability testing or separation, generally, and in particular, Percoll gradient processing. The use of fresh sperm in accordance with embodiments of the present invention herein may permit the use of low numbers of sperm for insemination even, for example, when using poor quality collected semen.

Alternative embodiments of the present invention may broadly provide for treatment, processing and preserving of insemination sperm. Alternative embodiments of the present invention may provide, for example, for the selection of the collected sperm cells more likely to achieve insemination. According to one embodiment of the present invention the selection of desired sperm cells or sperm cells more likely to achieve insemination may comprise concentrating the more motile sperm collected. Additional embodiments may providethe step of centrifuging the sperm through density gradients, and in preferred embodiments a Percoll gradient. A potentially preferred embodiment may use a 90:45% Percoll gradient. An additional embodiment may comprise limiting the concentration to less than about twice the starting concentration. Implementation of the Percoll gradient should be understood to encompass the use of Percoll pre or post processing of the sperm, and in particular, potentially pre or post sorting. Therefore, in accordance with embodiments herein, the Percoll gradient procedure may be used with "fresh sperm." The sample established from the desired or selected sperm cells may be used to establish the artificial insemination sample, potentially increasing the fertility rates due, at least in part, to the potentially higher rate of viability of the sample. The Percoll gradient procedure in accordance with the present invention may be conducted in conjunction with the use of lower numbers of sperm placed within the depositing catheter, as more particularly described supra.

Additionally, establishing an artificial insemination sample may be provided by establishing a sample having hysteroscopic compatible media, thereby providing for potentially increased fertility rates and an efficacious insemination procedure. More particularly, an embodiment of the invention may provide establishing an artificial insemination sample, and in preferred embodiments having a low number sperm compared to natural insemination doses, and providing for the establishment of an artificial insemination sample compatible media, for example extender, and in preferred embodiments, a skim milk medium, such as EZ-Mixin CST® (Animal Reproduction Systems, Chino, CA), preferably as a diluting media, potentially prior to additional processing. In accordance with preferred embodiments of the present invention, dilution may occur to no more than a 2:1 ratio, to no more than a 5:1 ratio, and to no more than a 10:1 ratio, to potentially achieve at least a hysteroscopic compatible volume or media with appropriate concentrations. Furthermore, embodiments of the present invention may provide for the use of an extender, potentially a second extender provided after sperm processing and potentially in conjunction with a first extender, as previously mentioned, to establish the sample utilizing hysteroscopic compatible media. The second extender may serve to resuspend the sperm sample after processing, and more particularly, provide for a sample utilizing hysteroscopic compatible media. According to preferred embodiments, the medium or second extender may comprise a TALP medium, a HEPES-buffered Tyrode"s medium, and an Androhep medium. In accordance with preferred embodiments of the present invention, dilution may be performed with a skim milk medium such as EZ-Mixin CST®, with a TALP medium, with a HEPES-buffered Tyrode"s medium, and with an Androhep medium, either single or in combination, to potentially achieve at least a hysteroscopic compatible volume or media with appropriate concentrations.

An additional embodiment of the present invention may also provide for an artificial insemination sample utilizing a hysteroscopic compatible media or medium having a catheter coordinated viscosity. The viscosity may, for example, potentially facilitate the steps of extruding and depositing the sample. Furthermore, in accordance with potentially preferred embodiments of the present invention, the use of compatible media or medium may create an artificial insemination sample having a viscosity of preferably greater than about that of the blood of said mammal or greater than about that of a saline solution. Furthermore additional embodiments may provide for compatible media having a viscosity of greater than about 100cp, a media having a viscosity of greater than about 300cp, a media having a viscosity of greater than about 1000cp, a media having a viscosity of greater than about 3000cp, and a media having a viscosity of greater than about 6000cp, each at about the mammal's average body temperature.

Furthermore, an embodiment of the present invention may provide a hysteroscopic insemination sample comprising a reservoir element, a catheter system to which said reservoir element is responsive, a plurality of sperm cells contained within said reservoir element; and hysteroscopic compatible media contained within said reservoir element and to which said sperm cells are responsive. In accordance with additional embodiments, an insemination containment wherein said plurality of sperm cells are contained within said reservoir element may include a low number of sperm cells as compared to the number of sperm cells typically found in a natural insemination.

Altering a property of the insemination specimen or sperm cell sample may be conducted according to the present invention, and in accordance with preferred embodiments, determining an estrous time for a female of a species of said mammal and then altering a property of said insemination specimen to establish an artificial insemination sample at about said estrous time. Alternative embodiments may provide altering a property of the sample at about the time determined for the female of said species to be appropriately fertile, as previously defined. The present invention may also provide initiation of the altering of a property of the insemination specimen within a time selected from: within about twenty-four hours of said time determined for said female of said species of said mammal to be appropriately fertile, within about twelve hours of said time determined for said female of said species of said mammal to be appropriately fertile, within about eight hours of said time determined for said female of said species of said mammal to be appropriately fertile, within about three hours of said time determined for said female of said species of said mammal to be appropriately fertile, and within about one hour of said time determined for said female of said species of said mammal to be appropriately fertile. This alteration may consist of preparing the sample, sorting the sperm, thawing the sperm, or the like.

One particular embodiment of the present invention may provide for establishing a hysteroscopic compatible media and the concentration of sperm to select sperm more likely to achieve insemination. Particularly, one potentially preferred embodiment of the invention may provide preparation of the semen through centrifugation. The semen may be diluted to provide, for example, 100 x 10⁶ spermatozoa/ml in preferably a commercial skim milk extender (EZ-Mixin CST®, Animal Reproduction Systems, Chino, CA). The sperm suspension may be protected from light and maintained for preferably 6 hours at room (20 - 25^{o}c) temperature to simulate the potential time needed to sort the spermatozoa, if so desired. The sperm suspension may then be centrifuged through a 90:45% Percoll (Sigma Chemical Co., St. Louis, MO, USA) discontinuous density gradient with the goal of reconcentrating the cells and to potentially select a highly motile fraction of spermatozoa. The 90% Percoll may be diluted at a ratio of 1:1 (v/v) with HEPES-buffered Tyrode"s medium (Grøndahl et al., 1996) to make a 45% solution. In a preferably 15-mL centrifuge tube, preferably 1 mL of 45% Percoll may be carefully layered on top of preferably 1 mL of 90% Percoll. Preferably 1 mL of the sperm suspension (100 x 10⁶ sperm/mL in EZ-Mixin, CST) may be layered on top of the Percoll layers, and the tube may be centrifuged at 800 x g for a preferred period of 12 minutes. After centrifugation, the supernatant may be completely removed and the pellet may be resuspended in preferably 600µl HEPES-buffered Tyrode's Medium. The sperm concentration may be determined, in accordance with one embodiment, using a Densimeter (534B MOD-1, Animal Reproduction Systems, Chino, CA) and the potential volume to deliver 5 million spermatozoa (potentially of a preferred ~100µl) may be calculated and prepared for insemination.

In accordance with a broad aspect of the present invention, one embodiment thereof may provide for the positioning of the catheter near the UTJ of the female species of mammal wherebythe catheter isinserted under a surface in the vicinity of the uterotubal junction. At least a portion of the sample may be extruded or deposited in the vicinity of the UTJ under the surface.

One embodiment of the present invention may provide for the collecting of sperm cells from the male species of mammal, establishing an artificial insemination sample utilizing at least some of the sperm cells collected, and placing the artificial insemination sample in a catheter. A determination of a time when the female is appropriately fertile may be determined, as described above. The optical element (12) and the catheter (2) may be inserted vaginallyand guided through the vagina, as depicted in Figure 2. The UTJ may be optically located with optical element (12).

The catheter may be inserted under a surface (34) in the vicinity of the UTJ (30), as depicted in Figure 3. At least a portion of said artificial insemination sample may be extruded under the surface within the vicinity of the UTJ, thus creating a "blister" with the sample enshrouded between layers. Deposition of at least a portion of the artificial insemination sample may be provided under the surface (34) in the vicinity of the UTJ. The surface (34) may comprise the endometrium or other portion of the uterus or the uterine lining. In accordance with one preferred embodiment, the catheter may be inserted such that a particular portion of the uterus is not pierced, and in accordance with one embodiment, such that a vascularized portion of the uterus is not pierced. A vascularized portion of the uterus that may not be pierced, according to one particular embodiment, may include, for example, the mesometrium or the myometrium portion of the uterus or other vascularized portions. Thereafter, the optical element and catheter may be withdrawn from the female and fertilization of an egg of the female may occur after which production of an offspring mammal may be expected from the fertilized egg.

An embodiment of the present invention may also provide a potentially corresponding insemination catheter having a guide element, or in preferred embodiments a videoendoscope or a cannula, a reservoir element responsive to the guide element, an extrusion element, or in preferred embodiments a syringe to which said reservoir element is responsive, and a cellular piercing tip (36) positioned in front of the reservoir element. The catheter may further provide a pierce depth control element, such as an adjustment element or a stop on the piercing tip (36) positioned in the vicinity of the tip. One embodiment of an insemination catheter of the present invention is depicted in Figure 3.

As previously described and in accordance with the insemination system of extrusion and deposition under a surface in the vicinity of the UTJ described above, a number of steps of producing a mammal may be performed. A low number of sperm may be placed in the catheter (2), and in preferred embodiments, preferably numbers selected from: less than about ten million sperm, less than about five million sperm, less than about two million sperm, less than about one million sperm, less than about five hundred thousand sperm, and less than about one hundred thousand sperm. Furthermore, the fertilization of an egg may be performed in accordance with the preferred embodiments of the invention wherein success levels success levels of fertilization may be statistically comparable to a conventional uterine body artificial insemination process. Statistically comparable success levels may be defined as previously mentioned.

Furthermore, the sperm cells may be collected from a male species of mammal, in alternative embodiments of the invention, comprising bovids, equids, or swine. In accordance with alternative embodiments of the invention, sperm cells may be selected from collected cells for those cells that may be more likely to achieve insemination, as previously described.

Accordingly, an insemination containment elementmay be provided, in accordance with embodiments of the invention, preferably comprising a cellular base surface, and in particular embodiments a uterine lining or, in accordance with preferred embodiments, a nonvascularized portion of the uterus, such as the mesometrium or the myometrium, a cellular cover surface adjacent to the cellular base surface, and in particular embodiments, the endometrium or uterine lining, a substantial enshrouded volume between the cellular base and the cover surface, and in preferred embodiments located in the vicinity of the UTJ, and sperm cells from the male of the species. Preferred embodiments may also utilize low numbers of sperm relative to natural insemination, located within the volume and a sperm emission element adjacent the volume through which sperm may pass. The sperm may be collected, selected, of an inseminate volume, perhaps even of an epididymis origin, or of any other limitation previously discussed. The sperm emission element may comprise a breach in the endometrium surface of the uterus, as depicted in Figure 3, or may simply occur by diffusion or the like.

Additionally, other potentially independent procedures may be incorporated into the present invention and may still be considered as within the scope of the present invention. Such procedures may include sorting the sperm cells by a sex characteristic, thereby establishing a sex-sorted artificial insemination sample, and in preferred embodiments having a low number of sperm compared to a natural insemination dosage for said mammal, may include establishing a low dose sex-selected artificial insemination sample. Furthermore, preserving or freezing, and the subsequent thawing of, sperm sells may be accomplished in particular embodiments, particularly in regard to various mammals such as equid, bovid and swine. Deposition of the insemination sample, may be processed or aspirated in any way, may be deposited with the crypts or folds of the UTJ, and may provide some type of preservation of the sperm for subsequent insemination. Establishment of an insemination specimen or insemination sample at a hysteroscopic compatible volume and utilizing compatible media may further provide for allowing cooling of the specimen or sample at room temperature. Centrifugation may preferably be performed through a Percoll gradient for about five minutes at about 200g and for about ten minutes at about 800g. In accordance with a preferred embodiment, concentrating the more motile sperm may be limited to concentrating to less than about twice the starting concentration. In particular, the broad and narrow concepts embodied in the present invention should be construed as applying to other species of mammal, including equids, bovids and swine. Finally the present invention, directed in part to the producing of an offspring mammal, may further be considered to disclose an embodiment of an animal produced utilizing a process as described in any of the foregoing method claims.

Sorting, in accordance with embodiments of the present invention, may particularly provide for collecting sperm cells from a male of a species of mammal, sorting the sperm cells according to a sex-specific characteristic, establishing a sorted, sex-specific artificial insemination sample, placing the sorted, sex-specific artificial insemination sample in a catheter; among the various other aspects of the invention disclosed herein that might be incorporated in method of producing a mammal.

As can be easily understood from the foregoing, the basic concepts of the present invention may be embodied in a variety of ways. It involves both insemination techniques as well as apparatus to accomplish appropriate insemination. In this application, the insemination techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the devices described, but also method or process claims may be included to address the functions the invention and each element performs. Neither the description nor the terminology is intended to limit the scope of the claims which will be included in a full patent application.

It should also be understood that a variety of changes may be made without departing from the essence of the invention. Such changes are also implicitly included in the description. They still fall within the scope of this invention.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms -- even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of an "extruder" should be understood to encompass disclosure of the act of "extruding"-- whether explicitly discussed or not -- and, conversely, were there only disclosure of the act of "extruding", such a disclosure should be understood to encompass disclosure of an "extruder" and even a "means for extruding". Such changes and alternative terms are to be understood to be explicitly included in the description.

Any acts of law, statutes, regulations, or rules mentioned in this application for patent, or patents, publications, or other references mentioned in this application for patent, are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in the Random House Webster's Unabridged Dictionary, second edition are hereby incorporated by reference. However, as to each of the above, to the extent that such information or statements incorporated by reference might be considered inconsistent with the patenting of this/these invention(s), such statements are expressly not to be considered as made by the applicant(s).

Thus, the applicant(s) should be understood to have support to claim at least: i) each of the insemination devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, and ix) methods and apparatuses substantially as described hereinbefore and with reference to any of the accompanying examples, and x) the various combinations and permutations of each of the elements disclosed.

Further, if or when used, the use of the transitional phrase "comprising" is used to maintain the "open-end" claims herein, according to traditional claim interpretation. Thus, unless the context requires otherwise, it should be understood that the term "comprise" or variations such as "comprises" or "comprising", are intended to imply the inclusion of a stated element or step or group of elements or steps but not the exclusion of any other element or step or group of elements or steps. Such terms should be interpreted in their most expansive form so as to afford the applicant the broadest coverage legally permissible.

I. PATENT DOCUMENTS

| | | | | | |
|---|---|---|---|---|---|
| DOCUMENT NO. | DATE | NAME/COUNTRY | CLASS | SUBCLASS | FILING DATE |
| 5,135,759 | Aug. 4, 1992 | Johnson | 424 | 561 | Apr. 26, 1991 |
| 6,071,689 | Jun. 6, 2000 | Seidel et al. | 435 | 2 | Jan. 29, 1998 |
| 60/238,294 | May 10, 2000 | Morris et al. | | | May 10, 2000 |
| WO 98/34094 | Jun. 8, 1998 | NZ | | | |
| WO 99/33956 | Aug. 7, 1999 | US | | | |
| WO 99/38883 | May 8, 1999 | US | | | |
| WO 99/42810 | Aug. 26, 1999 | US | | | |
| WO 00/06193 | Oct. 2, 2000 | US | | | |

II. OTHER DOCUMENTS (Including Author, Title, Date, Pertinent Pages, Etc.)

Brarher, V. and Allen, W. R., “Videoendoscopic Examination of the Mare's Uterus: Findings in Normal
Fertile Mares”, Equine Veterinary Journal, Vol. 24 (1992), pp. 274-278
Buchanan, B. R., et al, “Insemination of Mares with Low Numbers of Either Unsexed or Sexed
Spermatozoa”, Theriogenology, Vol. 53, pp 1333-1344, (2000)
Caslick, E. A., “The Vulva and the Vulvo-vaginal Orifice and its Relation to Genital Health of the
Thoroughbred Mare”, Cornell Veterinarian, Vol. 27, 1937, pp. 178-187
Cran, D. G., et al, “Production of Lambs by LowDose Intrauterine Insemination with Flow Cytometrically
Sorted and Unsorted Semen”, Theriogenology, Vol. 47, pp. 267, (Abstract), (1997)
Fugger, E. F., “Clinical Experience with Flow Cytometric Separation of Human X- and Y- Chromosome
Bearing Sperm”, Theriogenology, Vol. 52, pp. 1435-1440 (1999)
Grondahl, C., et al, “In Vitro Production of Equine Embryos”, Biology of Reproduction, Monograph Series
I, pp. 299-307 (1995)
Johnson, L. A. and Welch, G. R., “Sex Preselection: High-speed flow cytometric sorting of X and Y sperm
for maximum efficiency”, Theriogenology, Vol. 52, (1999), pp. 1323-1341
Johnson, L.. A., “Advances in Gender Preselection in Swine” Journal of Reproduction and Fertility
Supplement, Vol. 52, pp. 255-266 (1997)
Johnson, L.. A., “Sex Preselection in Swine: Altered Sex Ratios in Offspring Following Surgical
Insemination of Flow Sorted X- and Y- Bearing Sperm”, Reproduction in Domestic Animals, Vol. 26, pp.
309-314 (1991)
Johnson, L.. A., et al., “Sex Preselection in Rabbits: Live Births from X and Y Sperm Separated by DNA and
Cell Sorting” Biology of Reproduction, Vol. 41, pp. 199-203 (1989)
Manning S. T., et al., “Development of Hysteroscopic Insemination of the Uterine Tube in the Mare”,
Proceedings of the Annual Meeting of the Society for Theriogenology, 1998, pp. 84-85.
Morris, L. H., et al., “Hysteroscopic insemination of small numbers of spermatozoa at the uterotubal junction

of preovulatory mares”, Journal of Reproduction and Fertility, Vol. 118, pp. 95-100 (2000)
Parrish, J. J., et al., “Capacitation of bovine sperm by heparin”, Biology of Reproduction, Vol. 38, pp. 1171-
1180 (1988)
Peippo, J., et al., “Sex diagnosis of equine preimplantation embryos using the polymerase chain reaction”,
Theriogenology, Vol. 44 619-627 (1995)
Pickett GW, et al., “Management of the mare for maximum reproductive efficiency” Bulletin No. 6
Colorado State University, Ft. Collins CO. (1989)
Schenk, J. L., “Cryopreservation of flow-sorted bovine spermatozoa”, Theriogenology, Vol. 52, 1375-
1391 (1999)
Schmid R. L., et al, “Fertilization with Sexed Equine Spermatozoa Using Intracytoplasmic Sperm Injection
and Oviductal Insemination”, 7th International Symposium On Equine Reproduction, pp. 139 (Abstract)
(1998)
Seidel, G. E. Jr, et al., “Artificial Insemination of Heifers with Cooled, Unfrozen Sexed Semen”,
Theriogenology, Vol. 49 pp. 365 (Abstract) (1998)
Seidel, G. E. Jr, et al., “Insemination of Heifers with Sexed Sperm”, Theriogenology, Vol. 52, pp. 1407-
1421 (1999)
Squires, E. L., “Early Embryonic Loss” in Equine Diagnostic Ultrasonography, 1^{st Ed. pp 157-163 Eds}
Rantanen & McKinnon. Williams and Wilkins, Baltimore, Maryland (1998)
Squires, E. L.., et al, “Cooled and frozen stallion semen”, Bulletin No. 9, Colorado State University, Ft.
Collins, CO. (1999)
Vazquez, J., et al., “A. I. in Swine; New Strategy for Deep Insemination with Low Number of Spermatozoa
Using a Non-surgical Methodology”, 14^{th International Congress on Animal Reproduction, Vol. 2,}
Stockhlom, July, 2000, p. 289.
Vazquez, J., et al., “Nonsurgicial Utero-tubal Insemination in the Mare”, Proceedings Annual Meeting of the
Society for Theriogenology, 1998, pp. 82-83.
Vazquez, J., et al., “Successful Low-Dose Insemination by a Fiberoptic Endoscope Technique in the Sow”,
Proceedings Annual Conference of the International Embryo Transfer Society, Netherlands, Theriogenology,
Vol. 53, January, 2000, pp. 201.
Vazquez, J., et al., “Development of a Non-surgical Deep Intra Uterine Insemination Technique”, IV
International Conference on Boar Semen Preservation, Maryland, August, 1999, p 35 and photo of display
board.
Vazquez, J., et al., “Development of a Non-surgical Deep Intra Uterine Insemination Technique”, Boar
Semen Preservation IV, IVth International Conference on Boar Semen Preservation, Maryland, pp. 262-263.
Vazquez, J., et al., “Hypoosmotic Swelling Test as Predictor of the Membrane Integrity in Boar
Spermatozoa”, Boar Semen Preservation IV, IVth International Conference on Boar Semen Preservation,
Maryland, pp. 263.
Johnson, L., et al, “Sex Preselection in Swine: Flow Cytometric Sorting of X- and Y- Chromosome Bearing
Sperm to Produce Offspring”, Boar Semen Preservation IV, 2000, pp. 107-114.
Rath, D., et al., “Low Dose Insemination Technique in the Pig”, Boar Semen Preservation IV, 2000, pp. 115-
118.
Lindsey, A., et al., “Hysteroscopic Insemination of Mares with Nonfrozen Low-dose Unsexed or Sex-sorted
Spermatozoa”, currently unpublished, pp. 1-15.

What is Claimed is:

1.
What is claimed is:
143.A method of producing a mammal comprising the steps of:
collecting sperm cells from a male of a species of mammal;
establishing an artificial insemination sample from said collected sperm cells;
placing said artificial insemination sample in a catheter;
determining a time when a female of said species of said mammal is appropriately fertile;
vaginally inserting an optical element into said female of said species of said mammal;
vaginally inserting said catheter into a female of said species of said mammal;
guiding said optical element and said catheter through said vagina of said female of said species of said mammal;
visually locating a uterotubal junction within said female of said species of said mammal;
positioning said catheter in the vicinity of said uterotubal junction within said female of said species of said mammal;
extruding at least a portion of said artificial insemination sample from said catheter;
depositing at least a portion of said artificial insemination sample in the vicinity of said uterotubal junction within said female of said species of said mammal;
withdrawing said optical element and said catheter from said female of said species of said mammal;
fertilizing an egg of said female of said species of said mammal; and
producing an offspring mammal from said fertilized egg.

2. 144.A method of producing a mammal as described in claim 143 wherein said step of placing said artificial insemination sample in a catheter comprises the step of placing a low number of sperm in said catheter.

3. 145.A method of producing a mammal as described in claim 144 wherein said step of placing a low number of sperm in said catheter comprises the step of placing a number of sperm selected from a group consisting of: less than about ten million sperm, less than about five million sperm, less than about two million sperm, less than about one million sperm, less than about five hundred thousand sperm, and less than about one hundred thousand sperm.

4. 146.A method of producing a mammal as described in claim 145 wherein said step of fertilizing comprises the step of fertilizing an egg of said female of said species of said mammal with success levels statistically comparable to a conventional uterine body artificial insemination process with success levels determined from a group consisting of:
fertilizing an egg of said female of said species of said mammal with success levels statistically comparable to a conventional uterine body artificial insemination process with a statistical P value greater than or equal to at least about 0.05,
fertilizing an egg of said female of said species of said mammal with success levels statistically comparable to a conventional uterine body artificial insemination process using a 500 million equine sperm sample with a statistical P value greater than or equal to at least about 0.05,
fertilizing an egg of said female of said species of said mammal with success levels statistically comparable to a conventional uterine body artificial insemination process with a statistical βvalue greater than or equal to at least about 80%,
fertilizing an egg of said female of said species of said mammal with success levels statistically comparable to a conventional uterine body artificial insemination process with both a statistical P value greater than or equal to at least about 0.05 and a statistical βvalue greater than or equal to at least about 80%.

5. 147.A method of producing a mammal as described in claim 145 wherein said step of fertilizing comprises the step of fertilizing an egg of said female of said species of said mammal with success levels statistically comparable to a conventional uterine body artificial insemination process with success levels selected from a group consisting of: at least about 75% success rates, at least about 65% success rates, at least about 60% success rates, at least about 50% success rates, at least about 45% success rates, and at least about 90% of a conventional artificial insemination for that species.

6. 148.A method of producing a mammal as described in claim 147 wherein said step of fertilizing comprises the step of fertilizing an egg of said female of said species of said mammal with success levels statistically comparable to a conventional uterine body artificial insemination process with success levels determined from a group consisting of:
fertilizing an egg of said female of said species of said mammal with success levels statistically comparable to a conventional uterine body artificial insemination process with a statistical P value greater than or equal to at least about 0.05,
fertilizing an egg of said female of said species of said mammal with success levels statistically comparable to a conventional uterine body artificial insemination process using a 500 million equine sperm sample with a statistical P value greater than or equal to at least about 0.05,
fertilizing an egg of said female of said species of said mammal with success levels statistically comparable to a conventional uterine body artificial insemination process with a statistical βvalue greater than or equal to at least about 80%,
fertilizing an egg of said female of said species of said mammal with success levels statistically comparable to a conventional uterine body artificial insemination process with both a statistical P value greater than or equal to at least about 0.05 and a statistical βvalue greater than or equal to at least about 80%.

7. 149.A method of producing a mammal as described in claim 147 wherein said step of collecting sperm cells from a male of a species of mammal comprises the step of collecting sperm cells from a male of a species of a mammal selected from a group consisting of: bovids, equids, or swine.

8. 150.A method of producing a mammal as described in claim 143 wherein said step of collecting sperm cells from a male of a species of mammal comprises the step of collecting epididymal sperm cells from a male of a species of mammal
250. An hysteroscopic insemination sample, comprising:
a reservoir element;
a catheter system to which said reservoir element is responsive; and
a hysteroscopic compatible volume of sperm contained within said reservoir element.

9. 251.An hysteroscopic insemination sample as described in claim 250 wherein said hysteroscopic compatible volume of sperm contained within said reservoir element comprise a low number of sperm cells as compared to a the number of sperm cells typically found in a natural insemination.

10. 252.An hysteroscopic insemination sample as described in claim 251 wherein said low number of sperm cells as compared to a the number of sperm cells typically found in a natural insemination comprises a number of sperm cells selected from a group consisting of: less than about ten million sperm, less than about five million sperm, less than about two million sperm, less than about one million sperm, less than about five hundred thousand sperm, and less than about one hundred thousand sperm.

11. 253.An hysteroscopic insemination sample as described in claim 252 wherein said sperm cells comprise sperm cells from a male of a species of a mammal selected from a group consisting of: bovids, equids, or swine.

12. 254.A hysteroscopic insemination sample as described in claim 250 wherein said hysteroscopic compatible volume of sperm contained within said reservoir element comprises a volume selected from a group consisting of: between about 30 and 150 ul, less than about 500 ul, about 230 ul, and about 100 ul.

13. 255.An hysteroscopic insemination sample as described in claim 250 wherein said hysteroscopic compatible volume of sperm contained within said reservoir element comprise sex-selected sperm cells from a male of a species of mammal.

14. 256.An hysteroscopic insemination sample as described in claim 255 wherein said sex-selected sperm cells from a male of a species of mammal comprise sperm cells from a male of a species of a mammal selected from a group consisting of: bovids, equids, or swine.

15. 257.An hysterscopic insemination sample as described in claim 250, wherein said insemination containment element is configured to be positioned in the vicinity of a uterotubal junction of a female of a species of a mammal and deposited within the vicinity of the uterotubal junction within said female of said species of said mammal by visually locating the uterotubal junction.

16. 258.A method of producing a mammal as described in claim 143 wherein said step of fertilizing an egg of said female of said species of said mammal comprises the step of fertilizing an egg of said female of said species of said mammal with success levels statistically comparable to a conventional uterine body artificial insemination process.

17. 259.A method of producing a mammal as described in claim 143 and further comprising the steps of freezing at least some of said collected sperm cells and thawing at least some of said frozen collected sperm cells.

18. 260.A method of producing a mammal as described in claim 259 wherein said step of collecting sperm cells from a male of a species of mammal comprises the step of collecting sperm cells from a male of a species of a mammal selected from a group consisting of: bovids, equids, or swine.

19. 261.A method of producing a mammal as described in claims 259 or 260, further comprising the step of sorting said collected sperm cells according to a sex-specific characteristic.

20. 262.A method of producing a mammal as described in claim 143 wherein said step of establishing comprises establishing an artificial insemination sample utilizing fresh sperm from said collected sperm cells.

21. 263.A method of producing a mammal as described in claim 143, further comprising the step of selecting from said collected sperm cells desirable sperm more likely to achieve insemination and wherein said step of establishing comprises establishing an artificial insemination sample using said desirable sperm for insemination.

22. 264.A method of producing a mammal as described in claim 263 wherein said step of selecting comprises concentrating the more motile sperm collected from said male species of said mammal.

23. 265.A method of producing a mammal as described in claim 264 wherein said step of concentrating comprises centrifuging said sperm through a Percoll gradient.

24. 266.A method of producing a mammal as described in claim 143, further comprising the steps of inserting said catheter under a surface in the vicinity of said uterotubal junction within said female of said species of said mammal and wherein said step of extruding comprises extruding at least a portion of said artificial insemination sample from said catheter under said surface in the vicinity of said uterotubal junction within said female of said species of said mammal and wherein said step of depositing comprises depositing at least a portion of said artificial insemination sample under said surface in the vicinity of said uterotubal junction within said female of said species of said mammal within said female of said species of said mammal.

25. 267.A method of producing a mammal as described in claim 266 wherein said step of inserting comprises piercing an endomitrium surface of a uterus within said female of said species of said mammal.

26. 268.A method of producing a mammal as described in claim 267 wherein said step of inserting further comprises avoiding piercing a vascularized portion of said uterus of said female of said species of said mammal.

27. 269.A method of producing a mammal as described in claim 267 wherein said step of piercing an endomitrium surface comprises utilizing said catheter.

28. 270.A method of producing a mammal as described in claim 269, further comprising the step of controlling the depth to which said catether pierces said endomitrium surface of said uterus within said female of said species of said mammal.

29. 271.A method of producing a mammal as described in claim 270 wherein said step of controlling the depth comprises utilizing said catheter.

30. 272.A method of producing a mammal as described in claim 143, further comprising the step of aspirating at least a portion of said artificial insemination sample extruded from said catheter to create an aspirated insemination sample.

31. 273.A method of producing a mammal as described in claim 143, further comprising the step of determining a hysteroscopic insemination compatible volume for artificial insemination, wherein said step of establishing comprises establishing an artificial insemination sample at said hysteroscopic insemination compatible volume.

32. 274.A method of producing a mammal as described in claim 143, further comprising the steps of temporally substantially retaining a character of said collected sperm cells in an insemination specimen, determining an estrous time for said female of a species of said mammal, and altering a property of said insemination specimen to establish an artificial insemination sample at about said estrous time.

33. 275.A method of producing a mammal as described in claim 274, further comprising the step of initiating said step of altering a property of said insemination specimen to establish an artificial insemination sample at about said estrous time at about the time determined for said female of said species of said mammal to be appropriately fertile.

34. 276.A method of producing a mammal as described in claim 275 wherein said step of initiating comprises initiating said step of altering a property of said insemination specimen within a time selected from a group consisting of: within about twenty-four hours of said time determined for said female of said species of said mammal to be appropriately fertile, within about twenty-four hours of said time determined for said female of said species of said mammal to be appropriately fertile, within about twelve hours of said time determined for said female of said species of said mammal to be appropriately fertile, within about eight hours of said time determined for said female of said species of said mammal to be appropriately fertile, within about three hours of said time determined for said female of said species of said mammal to be appropriately fertile, and within about one hour of said time determined for said female of said species of said mammal to be appropriately fertile.

35. 277.A method of producing a mammal as described in claim 274, wherein said step of temporally substantially retaining a character of said sperm cells comprises the step of freezing said sperm cells and wherein said step of altering a property of said insemination specimen comprises the step of thawing said insemination specimen at about said estrous time.

36. 278.A method of producing a mammal as described in claim 143, further comprising the step of determining a time when a female of said species of said mammal is appropriately fertile, and wherein said step of establishing comprises establishing an artificial insemination sample having a low number of sperm compared to a natural insemination dosage for said mammal, wherein said step of depositing comprises depositing a low number of sperm in the vicinity of said uterotubal junction within said female of said species of said mammal, and wherein said step of fertilizing comprises fertilizing an egg of said female of said species of said mammal with success levels statistically comparable to a conventional uterine body artificial insemination process.

37. 279.A method of producing a mammal as described in claims 143, further comprising the step of sorting said collected sperm cells according to a sex-specific characteristic.

38. 280.A method of producing a mammal as described in claim 279 wherein said step of establishing comprises establishing a sorted, sex-specific artificial insemination sample, wherein said step of placing comprises placing said sorted, sex-specific artificial insemination sample in a catheter, wherein said step of extruding comprises extruding at least a portion of said sorted, sex-specific artificial insemination sample from said catheter, and wherein said step of depositing comprises depositing at least a portion of said sorted, sex-specific artificial insemination sample in the vicinity of said uterotubal junction within said female of said species of said mammal.

39. 281.A method of producing a mammal as described in claim 279 wherein said step of collecting sperm cells from a male of a species of mammal comprises the step of collecting sperm cells from a male of a species of a mammal selected from a group consisting of: bovids, equids, or swine.

40. 282.A method of producing a mammal as described in claims 150, 258, 259, 262, 263, 266, 272, 273, 274, 278, or 279 wherein said step of placing said artificial insemination sample in a catheter comprises the step of placing a low number of sperm in said catheter.

41. 283.A method of producing a mammal as described in claim 282 wherein said step of placing a low number of sperm in said catheter comprises the step of placing a number of sperm selected from a group consisting of: less than about ten million sperm, less than about five million sperm, less than about two million sperm, less than about one million sperm, less than about five hundred thousand sperm, and less than about one hundred thousand sperm.

42. 284.A method of producing a mammal as described in claim 283 wherein said step of fertilizing comprises the step of fertilizing an egg of said female of said species of said mammal with success levels statistically comparable to a conventional uterine body artificial insemination process with success levels determined from a group consisting of:
fertilizing an egg of said female of said species of said mammal with success levels statistically comparable to a conventional uterine body artificial insemination process with a statistical P value greater than or equal to at least about 0.05,
fertilizing an egg of said female of said species of said mammal with success levels statistically comparable to a conventional uterine body artificial insemination process using a 500 million equine sperm sample with a statistical P value greater than or equal to at least about 0.05,
fertilizing an egg of said female of said species of said mammal with success levels statistically comparable to a conventional uterine body artificial insemination process with a statistical βvalue greater than or equal to at least about 80%,
fertilizing an egg of said female of said species of said mammal with success levels statistically comparable to a conventional uterine body artificial insemination process with both a statistical P value greater than or equal to at least about 0.05 and a statistical βvalue greater than or equal to at least about 80%.

43. 285.A method of producing a mammal as described in claim 283 wherein said step of fertilizing comprises the step of fertilizing an egg of said female of said species of said mammal with success levels statistically comparable to a conventional uterine body artificial insemination process with success levels selected from a group consisting of: at least about 75% success rates, at least about 65% success rates, at least about 60% success rates, at least about 50% success rates, at least about 45% success rates, and at least about 90% of a conventional artificial insemination for that species.

44. 286.A method of producing a mammal as described in claim 285 wherein said step of collecting sperm cells from a male of a species of mammal comprises the step of collecting sperm cells from a male of a species of a mammal selected from a group consisting of: bovids, equids, or swine.

45. 287.A method of producing a mammal as described in claim 143 further comprising the step of determining a time when a female of said species of said mammal is appropriately fertile.

46. 288.A method of producing a mammal, comprising the steps of:
collecting sperm cells from a male of a species of mammal;
freezing at least some of said collected sperm cells;
thawing at least some of said frozen collected sperm cells;
sorting said collected sperm cells according to a sex-specific characteristic;
establishing an artificial insemination sample [utilizing fresh sperm] from said collected sperm cells having a low number of sperm compared to a natural insemination dosage for said mammal;
placing said artificial insemination sample in a catheter;
vaginally inserting an optical element into said female of said species of said mammal;
vaginally inserting said catheter into a female of said species of said mammal;
guiding said optical element and said catheter through said vagina of said female of said species of said mammal;
visually locating a uterotubal junction within said female of said species of said mammal;
positioning said catheter in the vicinity of said uterotubal junction within said female of said species of said mammal;
extruding at least a portion of said artificial insemination sample from said catheter;
depositing a low number of sperm of said artificial insemination sample in the vicinity of said uterotubal junction within said female of said species of said mammal;
withdrawing said optical element and said catheter from said female of said species of said mammal;
fertilizing an egg of said female of said species of said mammal with success levels statistically comparable to a conventional uterine body artificial insemination process; and
producing an offspring mammal from said fertilized egg.

47. 289.A method of producing a mammal as described in claim 288 wherein said step of collecting sperm cells from a male of a species of mammal comprises the step of collecting sperm cells from a male of a species of a mammal selected from a group consisting of: bovids, equids, or swine.

48. 290.A method of producing a mammal as described in claim 288 wherein said step of establishing comprises establishing a sorted, sex-specific artificial insemination sample, wherein said step of placing comprises placing said sorted, sex-specific artificial insemination sample in a catheter, wherein said step of extruding comprises extruding at least a portion of said sorted, sex-specific artificial insemination sample from said catheter, and wherein said step of depositing comprises depositing at least a portion of said sorted, sex-specific artificial insemination sample in the vicinity of said uterotubal junction within said female of said species of said mammal.

49. 291.A method of producing a mammal as described in claim 288, further comprising the step of determining a time when a female of said species of said mammal is appropriately fertile.

50. 292.A method of producing a mammal as described in claim 288 wherein said step of fertilizing an egg of said female of said species of said mammal comprises the step of fertilizing an egg of said female of said species of said mammal with success levels statistically comparable to a conventional uterine body artificial insemination process.

51. 293.A method of producing a mammal as described in claim 288 wherein said step of establishing comprises establishing an artificial insemination sample utilizing fresh sperm from said collected sperm cells.

52. 294.A method of producing a mammal as described in claim 288, further comprising the step of selecting from said collected sperm cells desirable sperm more likely to achieve insemination and wherein said step of establishing comprises establishing an artificial insemination sample using said desirable sperm for insemination.

53. 295.A method of producing a mammal as described in claim 288, further comprising the steps of inserting said catheter under a surface in the vicinity of said uterotubal junction within said female of said species of said mammal and wherein said step of extruding comprises extruding at least a portion of said artificial insemination sample from said catheter under said surface in the vicinity of said uterotubal junction within said female of said species of said mammal and wherein said step of depositing comprises depositing at least a portion of said artificial insemination sample under said surface in the vicinity of said uterotubal junction within said female of said species of said mammal within said female of said species of said mammal.

54. 296.A method of producing a mammal as described in claim 288, further comprising the step of aspirating at least a portion of said artificial insemination sample extruded from said catheter to create an aspirated insemination sample.

55. 297.A method of producing a mammal as described in claim 288, further comprising the step of determining a hysteroscopic insemination compatible volume for artificial insemination, wherein said step of establishing comprises establishing an artificial insemination sample at said hysteroscopic insemination compatible volume.

56. 298.A method of producing a mammal as described in claim 288, further comprising the steps of temporally substantially retaining a character of said collected sperm cells in an insemination specimen, determining an estrous time for said female of a species of said mammal, and altering a property of said insemination specimen to establish an artificial insemination sample at about said estrous time.

57. 299.A method of producing a mammal as described in claim 288 wherein said step of placing a low number of sperm in said catheter comprises the step of placing a number of sperm selected from a group consisting of: less than about ten million sperm, less than about five million sperm, less than about two million sperm, less than about one million sperm, less than about five hundred thousand sperm, and less than about one hundred thousand sperm.

58. 300.A method of producing a mammal as described in claim 299 wherein said step of fertilizing comprises the step of fertilizing an egg of said female of said species of said mammal with success levels statistically comparable to a conventional uterine body artificial insemination process with success levels determined from a group consisting of:
fertilizing an egg of said female of said species of said mammal with success levels statistically comparable to a conventional uterine body artificial insemination process with a statistical P value greater than or equal to at least about 0.05,
fertilizing an egg of said female of said species of said mammal with success levels statistically comparable to a conventional uterine body artificial insemination process using a 500 million equine sperm sample with a statistical P value greater than or equal to at least about 0.05,
fertilizing an egg of said female of said species of said mammal with success levels statistically comparable to a conventional uterine body artificial insemination process with a statistical βvalue greater than or equal to at least about 80%,
fertilizing an egg of said female of said species of said mammal with success levels statistically comparable to a conventional uterine body artificial insemination process with both a statistical P value greater than or equal to at least about 0.05 and a statistical β value greater than or equal to at least about 80%.

59. 301.A method of producing a mammal as described in claim 299 wherein said step of fertilizing comprises the step of fertilizing an egg of said female of said species of said mammal with success levels statistically comparable to a conventional uterine body artificial insemination process with success levels selected from a group consisting of: at least about 75% success rates, at least about 65% success rates, at least about 60% success rates, at least about 50% success rates, at least about 45% success rates, and at least about 90% of a conventional artificial insemination for that species.

60. 302.A method of producing a mammal as described in claim 301 wherein said step of collecting sperm cells from a male of a species of mammal comprises the step of collecting sperm cells from a male of a species of a mammal selected from a group consisting of: bovids, equids, or swine.

61. 303.A method of producing a mammal as described in claim 288 further comprising the step of determining a time when a female of said species of said mammal is appropriately fertile.

62. 304.An hysteroscopic insemination sample produced in accordance with the method of any of the foregoing claims.

63. 305.A mammal produced by the method of any of the foregoing claims.

64. 306.An insemination sample comprising a plurality of sperm cells contained within a reservoir element of a catheter system, wherein said sample is configured to be positioned in the vicinity of a uterotubal junction of a female of a species of a mammal and deposited within the vicinity of the uterotubal junction within said female of said species of said mammal by visually locating the uterotubal junction.

65. 307.An insemination sample as described in claim 306 wherein said sample comprises a hysteroscopic compatible media contained within said reservoir.

66. 308.An insemination sample as described in claim 306 wherein said sample comprises a hysteroscopic compatible volume of sperm contained within said reservoir.

67. 309.An insemination sample as described in claim 306 wherein said sample comprises a plurality of frozen thawed sperm cells.

68. 310.An insemination sample as described in claim 306 wherein said sample comprises a plurality of sorted sperm cells sorted according to a sex-specific characteristic.

69. 311.An insemination sample comprising a plurality of sperm cells, wherein said sample is configured to be positioned in the vicinity of a uterotubal junction of a female of a species of a mammal and deposited within the vicinity of the uterotubal junction within said female of said species of said mammal by visually locating the uterotubal junction.

70. 312.An insemination sample as described in claim 311 wherein said sample comprises a hysteroscopic compatible media.

71. 313.An insemination sample as described in claim 311 wherein said sample comprises a hysteroscopic compatible volume of sperm.

72. 314.An insemination sample as described in claim 311 wherein said sample comprises a plurality of frozen thawed sperm cells.

73. 315.An insemination sample as described in claim 311 wherein said sample comprises a plurality of sorted sperm cells sorted according to a sex-specific characteristic.
